(19) United States
(12) Patent Application Publication
(10) Pub. No.: US 2024/0418432 A1
(43) Pub. Date: Dec. 19, 2024

(54) Climate Chamber

(71) Applicant: Binder GmbH, Tuttlingen (DE)

(72) Inventors: Jochen Buschle, Nendingen (DE); Robin Kossmann, Tuttlingen (DE)

(73) Assignee: Binder GmbH, Tuttlingen (DE)

(21) Appl. No.: 18/740,253

(22) Filed: Jun. 11, 2024

(30) Foreign Application Priority Data
Jun. 13, 2023 (DE) 10 2023 115 363.3

(51) Int. Cl. F25D 17/04; F25D 17/06

(52) U.S. Cl. CPC F25D 17/042; F25D 17/06; F25D 2317/0413

(57) ABSTRACT

Provided is a climate chamber with an outer casing, with an inner casing disposed within the outer casing, and with a vapor inlet which penetrates a rear wall of the outer casing, in which in front of the vapor inlet in the region between the rear wall of the outer casing and an inner casing rear wall a flow barrier is disposed.

### CROSS-REFERENCE TO RELATED APPLICATION

The present application claims priority to German Patent Application No. 10 2023 115 363.3, filed Jun. 13, 2023, the entirety of which is incorporated herein by reference.

### FIELD OF APPLICATION

The application relates to climate chambers for storing items at defined temperatures and air humidity.

### BACKGROUND

Climate chambers are generally used in order to store items at defined temperatures and air humidity. To enable an increase in air humidity when this is necessary, vapor is conventionally introduced into the inner volume of the climate chamber through a vapor inlet.

This vapor inlet is often disposed centrally between the side walls of the climate chamber in its bottom region and penetrates the side located in the region of the backside, i.e. the side opposite the door or the doors of the climate chamber, to enable the uniform distribution of the moisture. The supplied vapor is intended to be dispersed between the inner and the outer casing and to enter through apertures in the walls of the inner casing, in particular in its side walls, into the interior of the inner casing.

However, it has been found in practice that herein the uniform distribution is only attained to a limited extent. Typically, a very moist air stream with vapor plumes spreads starting in the center of the climate chamber from the centrally disposed vapor inlet that only gradually reaches into the side portions of the climate chamber entailing the significant probability for an undesirable condensation in the proximity of the door.

### SUMMARY

The application addresses the problem of providing a climate chamber with an improved distribution of the moisture supplied through its vapor inlet. This problem is resolved through a climate chamber as disclosed herein.

The climate chamber according to the application has an outer casing, an inner casing disposed within the outer casing and with a vapor inlet that penetrates a rear wall of the outer casing. The vapor inlet is preferably fed from a vapor generator, dedicated to the climate chamber, in particular belonging to the climate chamber, with which it is connected through a piping system.

The essence of the application includes that upstream of the vapor inlet in the proximity between the rear wall of the outer casing and the inner casing rear wall a flow barrier is disposed. Accordingly, the flow barrier is not disposed directly in front of the vapor inlet, yet, seen from the direction of the door of the climate chamber it is located further removed from this door than the inner casing rear wall and closer to this door than the rear wall of the outer casing.

A flow barrier disposed in this region effects that the vapor stream cannot propagate unhindered substantially along in its forward direction into the inner volume of the climate chamber and thus toward the door, but first impacts the flow barrier and is deflected and/or vortexed by it. The thorough mixing of the vapor with the air and its distribution in the air are significantly improved.

Such flow barrier can especially simply be realized if the flow barrier is cuboidal. For example a plate of a suitable material, for example preferably of the same material as the inner casing, can then be utilized as the flow barrier. The installation of a cuboidal flow barrier is, in addition, very simple; oriented parallel to the rear wall of the outer casing it can be simply secured—preferably on the bottom of the outer casing.

According to an embodiment of the application, the height of the flow barrier corresponds to the distance between the bottom of the outer casing and the inner casing bottom and that the flow barrier is disposed on the bottom of the outer casing. In this way a direct stream of air, that, due to its high vapor content, is very moist, in the direction toward the door of the climate chamber is effectively prevented, even if the vapor exits relatively nondirectionally from the vapor inlet.

It is moreover especially preferred for the climate chamber to comprise at least one ventilator for drawing in air, in particular from the interior volume of the climate chamber. With the aid of such ventilator, recirculation of the air in the interior volume of the climate chamber can be effected which promotes the distribution of the vapor.

If the climate chamber comprises a vaporizer plate which is disposed in the region between the inner casing rear wall and the rear wall of the outer casing, it can contribute to the targeted regulation of the moisture in the climate chamber.

In climate chambers with ventilators and vaporizer plate it can be advantageous to dispose the vaporizer plate underneath the at least one ventilator in the region between the inner casing rear wall and the rear wall of the outer casing disposed such that air drawn in by the at least one ventilator is guided along between the inner casing rear wall and the vaporizer plate as well as also between the vaporizer plate and the rear wall of the outer casing.

Two air streams are thereby generated which initially have different degrees of humidity. The air stream which is guided in the air channel between the vaporizer plate and the rear wall of the outer casing is thoroughly mixed with the vapor which, after its impact on the flow barrier and vortexed by it, thereby obtains a relatively high degree of humidity. This moist air stream is guided on both sides laterally past the flow barrier and underneath the evaporator plate and is thereby guided directly into the side regions of the climate chamber, from where it arrives in particular through apertures in the side walls of the inner casing into the interior volume proper of the climate chamber and adjusts the desired degree of humidity.

In contrast, the air stream which is guided in the air channel between the vaporizer plate and along the inner casing rear wall, is marginally mixed with vapor and rather forms a relatively dry air curtain. In the central region of the climate chamber this applies also in the region that is located opposite to the vapor inlet since this region is shielded against the vapor by the flow barrier. Since in the side regions of the climate chamber already flows the moist air stream channeled through the open regions between the margins of the flow barrier and the side walls of the outer casing, the dry air is conducted further in the direction toward the door on which accordingly the condensation formation is especially effectively avoided.

This effect is enhanced thereby that the vaporizer plate is placed directly adjoining the flow barrier and/or that the vaporizer plate is aligned with the flow barrier.

### BRIEF DESCRIPTION OF THE DRAWINGS

FIG. 1 an exterior view of a climate chamber,

FIG. 2 a view onto a cutout of a cross section through the climate chamber, which extends along the center plane A perpendicular to the rear wall of the climate chamber,

FIG. 3 a view onto a cross section through the climate chamber that extends along the plane B extending parallel to the bottom of the climate chamber, wherein the cross section shows the climate chamber with the bottom of the inner casing removed, and

FIG. 4 a view onto a cross section through the climate chamber that extends along a plane C extending parallel to the rear wall of the climate chamber, which plane C lies behind the rear wall of the inner casing.

### DETAILED DESCRIPTION

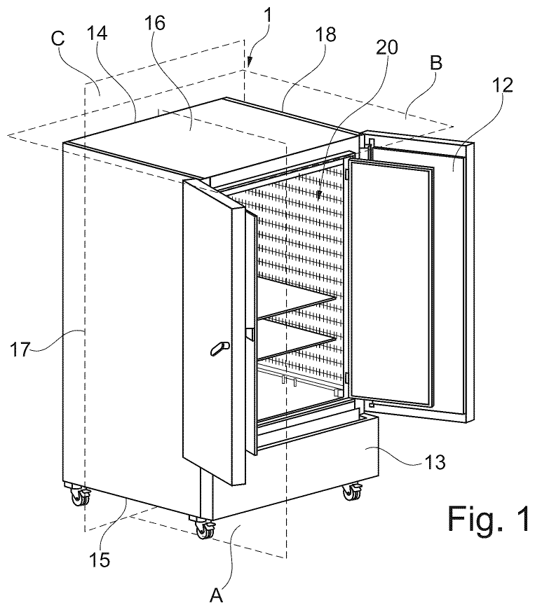

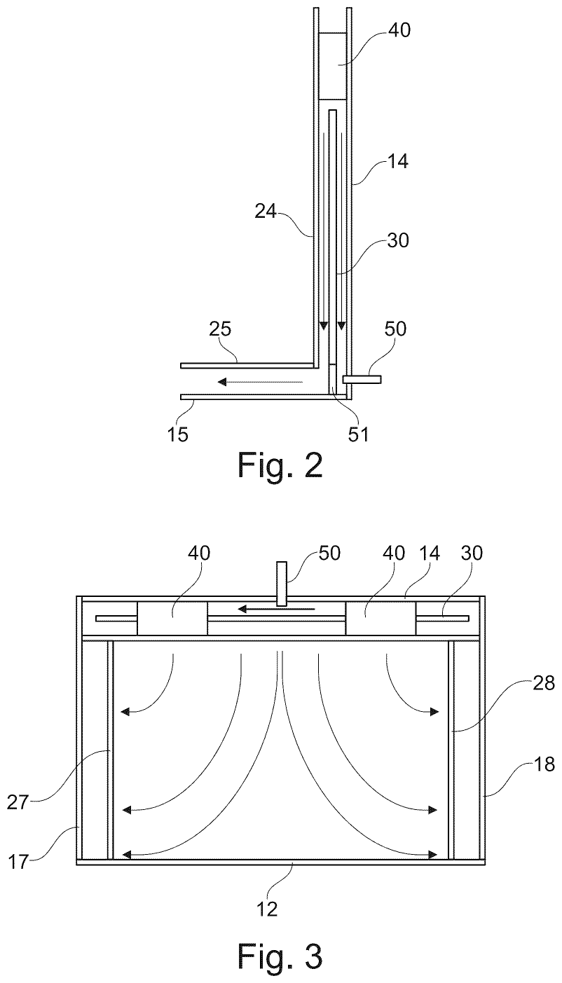

FIG. 1 shows an exterior view of a climate chamber 1. The climate chamber 1 is substantially cuboidal, wherein the outer surfaces of the cuboid, which together form the outer casing 10, by which are formed the front wall 13, identifiable by the door 12 disposed therein, the rear wall 14 opposite the front wall 13, a bottom 15, a ceiling 16, as well through the left wall face 17 and right wall face 18 of the climate chamber. FIG. 1 shows furthermore three sectional planes A, B and C which illustrate which views are shown in FIGS. 2 to 4 described in the following.

In addition to the outer casing 10, the climate chamber 1 comprises also an inner casing 20 which has at least an inner casing rear wall 24, an inner casing bottom 25 as well as inner casing side walls 27, 28 as can be seen in particular in FIGS. 2 and 3.

An overall integrated view of FIGS. 2 to 4 shows that between the rear wall 14 of the outer casing 10 and the inner casing rear wall 24 a vaporizer plate 30 is provided which is disposed substantially parallel to these walls. The lower edge of the vaporizer plate 30 terminates approximately at the height of the inner casing bottom 25. Moreover, the vaporizer plate 30 projects beyond the inner casing side walls 27, 28, in each instance toward the side.

Above the vaporizer plate 30 are provided two ventilators 40 with which an air stream directed substantially in the direction toward the bottom 15 can be generated, which air stream is illustrated in FIG. 2 by arrows. The air required for this purpose is preferably at least partially drawn in from the interior volume of the climate chamber 1 through apertures in the inner casing rear wall 24.

In the proximity of bottom 15 of the outer casing 10, stated more precisely in the region between the bottom 15 of the outer casing 10 and the inner casing bottom 25, a vapor inlet 50 is provided in the rear wall 14 of the outer casing 10. In front of the vapor inlet 50, but spaced apart therefrom, a flow barrier 51, which in this example is formed by a cuboidal plate, is disposed and specifically such that it is aligned with the vaporizer plate 30. To this end the thickness of the flow barrier 51 is adapted to the thickness of the vaporizer plate 30 and the flow barrier 51 is disposed at the same distance from the rear wall 14 of the outer casing 10 as is the vaporizer plate 30.

The height of the flow barrier 51, which directly adjoins the vaporizer plate 30 in the direction toward the bottom 15 of the outer casing 10, corresponds substantially to the distance between the inner casing bottom 25 and the bottom 15 of the outer casing 10.

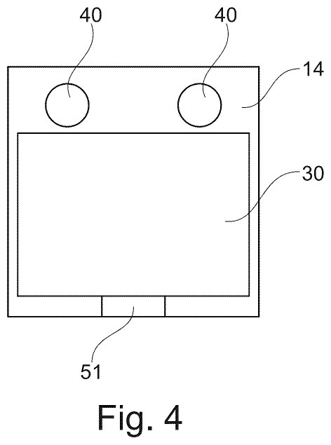

The width of the flow barrier 51, as can be recognized in particular in FIG. 4, is less than the distance of the ventilators 40 from one another and, in particular, less than the distance of the left wall face 17 of outer casing 10 from its right wall face 18. Accordingly, air flowing between the vaporizer plate 30 and the rear wall 14 of the outer casing 10 can exit to the right and left next to the flow barrier 51.

As FIG. 4 shows the flow barrier 51 is disposed approximately centrally. However, it can also be disposed at an offset which may be useful in particular if the ventilators 40 impart an angular momentum onto the air stream.

In particular the depiction according to FIG. 2 illustrates schematically the flow course of air conveyed by ventilators 40 into the volume between the inner casing rear wall 24 and the rear wall 14 of the outer casing 10. This air flows downwardly in front of and behind the vaporizer plate 30 in the direction toward the bottom 15 of the outer casing 10. In particular in the flow channel between the rear wall 14 of the outer casing 10 and the vaporizer plate 30, vigorous mixing occurs with the vapor fed in through the vapor inlet 50 since the vapor fed in through the vapor inlet 59 after it enters the volume between inner casing rear wall 24 and rear wall 14 of the outer casing 10 impinges onto the flow barrier 51 and is vortexed. In particular, the flow barrier 51 prevents therewith the direct propagation of vapor in the direction toward the door 12 and thus reduces the complex of condensation problems in the door region.

A further desirable effect of the flow barrier 51 following from the above is that the air mixed with the vapor is guided substantially laterally past the flow barrier 51. In particular, if this air is intended to be guided across apertures in the sidewalls 27, 28 of the inner casing 20 into the interior volume of the climate chamber 1 provided for storage, the stream of air mixed with vapor is guided in this manner relatively directly to the sites where this entrance is to take place.

In contrast to the air that flows along on the back side of the vaporizer plate 30, in the case of the air that flows in the flow channel between the inner casing rear wall 24 and the vaporizer plate 30, marginal interaction with vapor occurs since the direct access of the vapor to this region is blocked by the vaporizer plate 30 and the flow barrier 51. In particular in the central region a veil of dry air is thereby generated which is transported in the direction toward the door 12 and reliably prevents condensation on door 12.

### LIST OF REFERENCE SYMBOLS

1 Climate chamber
10 Outer casing
12 Door
13 Front wall
14 Rear wall
15 Bottom
16 Ceiling
17 Left wall face
18 Right wall face
20 Inner casing
24 Inner casing rear wall
25 Inner casing bottom
27, 28 Inner casing side wall
30 Vaporizer plate
40 Ventilator
50 Vapor inlet
51 Flow barrier
A, B, C Sectional plane

What is claimed:

1. A climate chamber comprising:
an outer casing;
an inner casing disposed within the outer casing;
a vapor inlet penetrating a rear wall of the outer casing; and
a flow barrier is disposed in front of the vapor inlet in a region between the rear wall of the outer casing and an inner casing rear wall.

2. The climate chamber of claim 1, wherein the flow barrier is cuboidal.

3. The climate chamber of claim 1, wherein a height of the flow barrier corresponds to a distance between a bottom of the outer casing and a bottom of the inner casing, wherein the flow barrier is disposed on a bottom of the outer casing.

4. The climate chamber of claim 1, wherein the climate chamber further comprises at least one ventilator for drawing in air from an interior volume of the climate chamber.

5. The climate chamber of claim 1, wherein the climate chamber further comprises a vaporizer plate disposed in a region between a rear wall of the inner casing and a rear wall of the outer casing.

6. The climate chamber of claim 5, wherein the climate chamber further comprises at least one ventilator for drawing in air from an interior volume of the climate chamber, wherein the vaporizer plate is disposed underneath the at least one ventilator in the region between the rear wall of the inner casing and the rear wall of the outer casing, wherein the air drawn in by the at least one ventilator is guided between the rear wall of the inner casing and the vaporizer plate as well as between the vaporizer plate and the rear wall of the outer casing.

7. The climate chamber of claim 5, wherein the vaporizer plate directly adjoins the flow barrier.

8. The climate chamber of claim 5, wherein that the vaporizer plate is aligned with the flow barrier.
